(19) United States
(12) Patent Application Publication
(10) Pub. No.: US 2024/0389878 A1
(43) Pub. Date: Nov. 28, 2024

(54) IN-VIVO VISUALIZATION DEVICE

(71) Applicant: NATIONAL UNIVERSITY CORPORATION CHIBA UNIVERSITY, Chiba-shi (JP)

(72) Inventors: Masahiro TAKEI, Chiba-shi (JP); Ryoma OGAWA, Chiba-shi (JP)

(21) Appl. No.: 18/524,823

(22) Filed: Nov. 30, 2023

(30) Foreign Application Priority Data
May 24, 2023 (JP) 2023-085482

(51) Int. Cl. A61B 5/0537; A61B 5/00; A61B 5/0536; A61B 5/251

(52) U.S. Cl. CPC A61B 5/0537; A61B 5/0536; A61B 5/251; A61B 5/7282; A61B 2562/0209

(57) ABSTRACT

To provide an in-vivo visualization device able to identify local changes in time and space.

An in-vivo visualization device according to an embodiment is provided with a current/voltage injection measurement unit which has a sensor provided with a plurality of electrodes arrangeable on a subject's skin at intervals from each other and which injects a current or applies a potential difference between each of the electrodes in a state where each of the electrodes is in contact with the skin and measures first measurement data, which is a potential difference and phase, based on a current injection/voltage measurement pattern when injecting the current, or measures second measurement data, which is a current and phase, based on a voltage injection/current measurement pattern when applying the potential difference between each of the electrodes, an image reconstruction unit which creates an electrical property distribution inside the subject's body based on the first measurement data or the second measurement data and predetermined parameters, and a region of interest identification unit that performs an identification process on the electrical property distribution to identify a region of interest and creates a post-identification electrical property distribution.

### BACKGROUND OF THE INVENTION

### Field of the Invention

The present invention relates to an in-vivo visualization device. Priority is claimed on Japanese Patent Application No. 2023-085482, filed May 24, 2023, the content of which is incorporated herein by reference.

### Background

In the medical, nursing, and healthcare fields, diseases associated with aging and mental stress have become major social issues. In particular, edema, a condition commonly seen with the diseases of the elderly, and sarcopenia, where a decrease in muscle mass is seen associated with aging, cause significant social harm. The importance of visualizing abnormalities, etc., in body fluids and tissues in-vivo in order to evaluate the states of these diseases is emphasized in the medical, nursing, and healthcare fields.

For example, methods such as X-ray computed tomography (CT), nuclear magnetic resonance imaging (MRI) are used in devices for visualizing conditions such as abnormalities in body fluids and tissues in-vivo to evaluate disorders. However, X-ray CT and MRI devices are extremely expensive and large, and ensuring appropriate imaging conditions and taking safety measures involves cost and effort. Thus, the development of a simple method for in-vivo visualization is a challenge.

As an example of a simple in-vivo visualization device, Japanese Patent No. 6555715 discloses a lymphedema monitoring device having an information processing device provided with an electrode band provided with a plurality of electrodes arranged on the periphery of a user's arms or legs, a measurement unit for measuring the electrical characteristics relating to the relaxation frequency specific to albumin, and a lymphedema estimation unit for detecting early lymphedema based on the electrical characteristics measured by the measurement unit.

### SUMMARY OF THE INVENTION

According to the measurement technique disclosed in Japanese Patent No. 6555715, it is possible to obtain the amount and a low-resolution distribution of body fluids and tissues included in a cross-section of measurement, but it is not possible to obtain said distribution at a resolution high enough to evaluate the state of a disease. That is, the spatial distribution of body fluids and tissues within a living body is complex, and regardless of any disorders in the subject, the changes of body fluids and related substances are significant even during a short period of time. Thus, there is a demand for an in-vivo visualizing technique able to identify local changes in time and space in more detail than the measurement technique of Japanese Patent No. 6555715.

The present invention was created in view of the above circumstances and provides a simple in-vivo visualization device able to identify local changes in time and space with high precision.

In order to solve the above problems, the present invention proposes the following aspects:

<1> An in-vivo visualization device of a first aspect of the present invention includes a current/voltage injection measurement unit which has a sensor provided with a plurality of electrodes arrangeable on a subject's skin at intervals from each other and which injects a current or applies a potential difference between each of the electrodes in a state where each of the electrodes is in contact with the skin and measures first measurement data, which is a potential difference and phase, based on a current injection/voltage measurement pattern when injecting the current, or measures second measurement data, which is a current and phase, based on a voltage injection/current measurement pattern when applying the potential difference between each of the electrodes, an image reconstruction unit which creates an electrical property distribution inside the subject's body based on the first measurement data or the second measurement data and predetermined parameters, and a region of interest identification unit that performs an identification process on the electrical property distribution to identify a region of interest and creates a post-identification electrical property distribution.

<2> A second aspect of the present invention is the in-vivo visualization device of the first aspect, in which the identification process may be performed based on a predetermined threshold.

<3> A third aspect of the present invention is the in-vivo visualization device of the first aspect or second aspect, in which the region of interest identification unit may create a region of interest index distribution by performing a standard deviation process on the electrical property distribution and create the post-identification electrical property distribution by performing the identification process on the region of interest index distribution.

<4> A fourth aspect of the present invention is the in-vivo visualization device of the first aspect or second aspect, in which the image reconstruction unit may create a multiple measurement matrix from at least one of impedance, resistance, reactance, capacitance, admittance, conductance, susceptance, and phase obtained from the first measurement data or the second measurement data, and create the electrical property distribution from the multiple measurement matrix and the predetermined parameters, and the predetermined parameters may include a prior variance vector γ relating to sparsity, a positive definite matrix B relating to temporal correlation, and a variance value λ of a multidimensional normal distribution relating to noise.

<5> A fifth aspect of the present invention is the in-vivo visualization device of the first aspect, which may further include a learning unit that updates the predetermined parameters based on at least the post-identification electrical property distribution.

<6> A sixth aspect of the present invention is the in-vivo visualization device of the fifth aspect, in which the learning unit may update the predetermined parameters by maximizing an average data likelihood using an expectation-maximization algorithm.

Each of the above aspects of the present invention provides a non-invasive in-vivo visualization device which can replace X-ray CT devices or MRI devices. In addition, since the in-vivo visualization device can carry out identification processes and use optimized parameters related to sparsity, temporal correlation, and noise, it can identify local changes in time and space, which was difficult with the prior art techniques. In addition, by evaluating the local states of body fluids and tissues within a living body, the device can help the diagnosis of various diseases and health conditions.

### BRIEF DESCRIPTION OF THE DRAWINGS

FIG. 1 is a schematic diagram of an internal skin measurement device according to an embodiment.

FIG. 2 is a schematic diagram of a current/voltage injection measurement unit in FIG. 1.

FIG. 3A is a diagram for showing a current injection/voltage measurement pattern using the counter electrode method.

FIG. 3B is a diagram for showing a current injection/voltage measurement pattern using the adjacent method.

FIG. 3C is a diagram for showing a current injection/voltage measurement pattern using the reference method.

FIG. 4 is a flowchart of an in-vivo visualization method according to the present embodiment.

FIG. 5A is a simple simulated image of a human calf.

FIG. 5B is a diagram showing a lymphedema simulating condition of Condition 1.

FIG. 5C is a diagram showing a venous edema simulating condition of Condition 2.

FIG. 6 is a diagram showing a conductivity distribution Δσ which was image-reconstructed in an Example, a Comparative Example, and a Prior Art Example under Condition 1.

FIG. 7 is a diagram showing the conductivity distribution Δσ which was image-reconstructed in the Example, the Comparative Example, and the Prior Art Example under Condition 2.

FIG. 8 is a schematic diagram of the measurement device used in Experiment Example 2.

FIG. 9 is a diagram for showing the experimental protocol relating to prolonged standing and leg elevation for inducing local spatiotemporal changes (LSTC) in extracellular fluid (ECF) of subcutaneous adipose tissue (SAT).

FIG. 10 is a frequency difference conductivity distribution Δσ of a certain subject reconstructed by an in-vivo visualization device.

FIG. 11A is a cross-sectional view of a typical human calf.

FIG. 11B shows Δσ of subject no. 12.

FIG. 11C is an ultrasound image in the vicinity of EIT electrode no. (4) of the subject.

FIG. 12 is a diagram showing a normalized spatial-mean conductivity <Δσ>^{SAT} in separated SAT and a normalized conventional BIA impedance z^{BIA} during prolonged standing and leg elevation.

FIG. 13 is a diagram showing the correlation between the spatial-mean conductivity <Δσ>^{SAT} of the separated SAT and the conventional impedance z^{BIA} for the calf of a certain subject.

FIG. 14 is a diagram showing changes over time in ΔL^{ECF} for the right legs of 15 subjects.

FIG. 15 is a diagram showing the correlation between the maximum volume change (ΔL^{ECF} at t8=40 minutes) in ECFmax (ΔL^{ECF}) measured by bioelectrical impedance analysis of the prior art (conventional BIA) in 15 subjects and the muscle mass of the right leg v^{RL}.

FIG. 16A is an example of a human calf including muscles, SAT, and the GSV.

FIG. 16B is a diagram showing conditions for a conductivity value σ for simulation.

FIG. 17A shows Δσ at iter=15 which was image-reconstructed by each algorithm against an unstable background field.

FIG. 17B is an ideal reconstructed image.

FIG. 17C shows the corresponding normalized spatial-mean conductivity <Δσ^{˜}k>^{perturb}.

FIG. 17D shows the <LE>, SE, and IN of the Example, Comparative example, and Prior Art Example.

### DETAILED DESCRIPTION OF THE INVENTION

The following description explains an in-vivo visualization device 100 according to an embodiment of the present invention with reference to the drawings. As shown in FIG. 1, the in-vivo visualization device 100 is provided with a current/voltage injection measurement unit 10 and a measurement calculation unit 50. The measurement calculation unit 50 is provided with an image reconstruction unit 52, a region of interest identification unit 53, a learning unit 54, and an output unit 55. The measurement calculation unit 50 of the in-vivo visualization device 100 is provided with, for example, a Central Processing Unit (CPU), a Read Only Memory (ROM), a Random Access Memory (RAM), and a Hard Disk Drive (HDD)/Solid State Drive (SSD). The image reconstruction unit 52, the region of interest identification unit 53, the learning unit 54, and the output unit 55 are realized by executing a predetermined program in the CPU. The program may be acquired via a recording medium or may be acquired via a network. In addition, a dedicated hardware configuration may be used to realize the configuration of the in-vivo visualization device 100. The following description will explain each unit.

### (Current/Voltage Injection Measurement Unit 10)

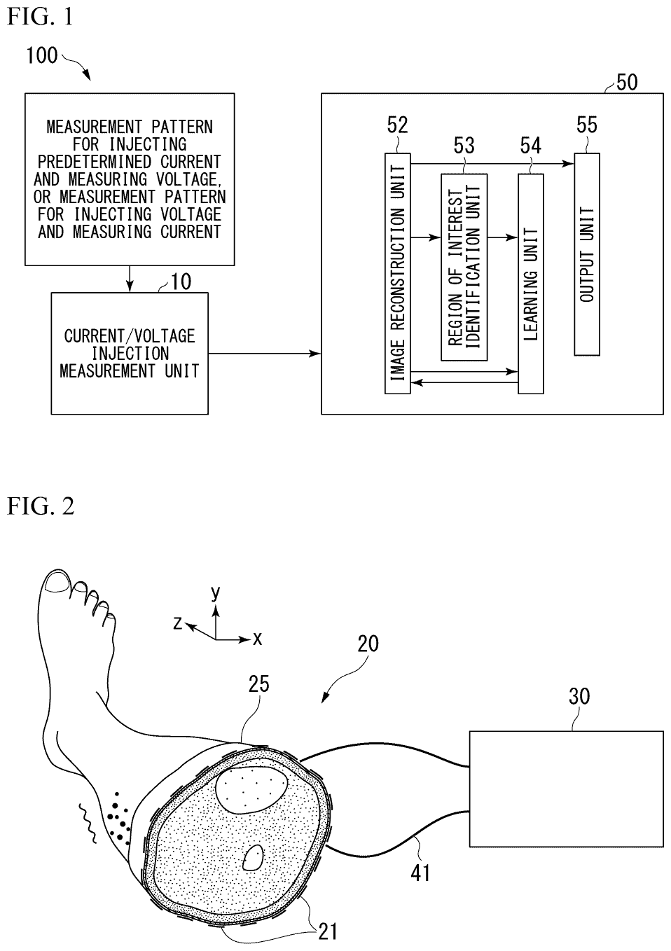

The following description explains the current/voltage injection measurement unit 10 using FIG. 2. FIG. 2 is a schematic diagram of the current/voltage injection measurement unit 10 in a state where a sensor 20 is arranged on a leg of a subject. As shown in FIG. 2, the current/voltage injection measurement unit 10 is provided with one or more of the sensors 20 and a control unit 30. The sensor 20 is provided with a plurality of electrodes 21 (for example, the number Q of the electrodes at 16 as shown in the diagram) able to be arranged on the skin at intervals from each other and a support body 25 that holds the electrodes 21. The current/voltage injection measurement unit 10 uses one or more of the sensors 20, injects a predetermined current or applies potential difference between each of the electrodes 21 in a state where each of the electrodes 21 of the sensor 20 is in contact with the skin of the person to be measured, and measures the potential difference or current. Here, the potential difference represents the difference in voltage between two electrodes. In a case of injecting a current, the potential difference is measured based on a predetermined current injection/voltage measurement pattern (in which two electrodes are sequentially selected from a large number of electrodes, a current is injected, and the potential difference is sequentially measured). At this time, it is desirable to also measure the phase (i.e., the time lag between the injected current and the measured potential difference). In a case of applying a potential difference, the current is measured based on a predetermined voltage injection/current measurement pattern (in which two electrodes are sequentially selected from a large number of electrodes, a potential difference is applied, and the current is sequentially measured). At this time, it is preferable to also measure the phase (i.e., the time lag between the applied potential difference and the measured current). Below, the description will focus on a case in which a current is injected and detailed description of a case in which a potential difference is applied may be omitted. Here, the number Q of the electrodes 21 is, for example, 3 or more. The number Q of the electrodes 21 is preferably 8 or more. The number Q of the electrodes 21 is preferably 32 or less. The number Q of the electrodes 21 may be 16 or less. In order to improve the precision of the concentration distribution, it is preferable to have a large number of electrodes. The arrangement positions of the electrodes 21 are not particularly limited. The electrodes 15 are preferably arranged at even intervals to encircle the periphery of the subject (here, the periphery of the leg).

The electrodes 21 are electrically connected to the control unit 30. The materials and shapes of the electrodes 21 are not particularly limited as long as it is possible to inject or apply a current or apply potential difference to the skin of the person to be measured. Examples of the material of the electrodes 21 include metals such as Au, Ag. Cu, and stainless steel, conductive polymers, fibers having surfaces coated with metal, fibers having surfaces coated with conductive substances such as conductive polymers. In order to bring each of the electrodes 21 into contact with the skin, the shape of the electrodes 21 in a plane orthogonal to the current injection direction is not particularly limited, but may be, for example, a circular or polygonal shape. The electrodes 21 are preferably non-invasive electrodes.

The electrical connection method between the electrodes 21 and the control unit 30 is not particularly limited and it is possible to use any known electrical connection method. In the present embodiment, each of the electrodes 21 is connected by an electrical wire 41 to the control unit 30.

The support body 25 is not particularly limited other than being able to hold the electrodes 21. Due to the support body 25, the electrodes 21 can be arranged on the skin of the subject, which is preferable. The term “can be arranged on the skin of the subject” means that each of the electrodes 21 is arranged to come into contact with the skin when the sensor 20 is worn by the subject.

Preferably, it is possible to apply a predetermined pressure to the support body 25 such that it is possible to bring the electrodes 21 into close contact with the subject. Due to this, it is possible to improve the adhesion between the electrodes 21 and the subject and more accurately inject a current or apply potential difference and measure the potential difference or current. Without being particularly limited, as the material of the support body 25, for example, insulators such as elastomers, leather, and cloth are preferable. The shape of the support body 25 is not particularly limited and examples thereof include a band shape or the like.

### (Control Unit)

The control unit 30 is provided with, for example, a multiplexer for switching between a current injection electrode that injects a current (or a voltage injection electrode that applies a potential difference) and a voltage measuring electrode that measures a potential difference (or a current measuring electrode that measures a current), an impedance analyzer that performs voltage measurement (or current measurement) and phase measurement, etc. An impedance analyzer is a component that measures impedance, that is, the ratio of a measured potential difference (applied potential difference) to an injected current (measured current) and the phase thereof by changing the applied frequency and amplitude. For example, the control unit 30 executes a predetermined program in the CPU and controls the multiplexer and the impedance analyzer to perform impedance measurement (measurement of the ratio of the potential difference to the current and the phase thereof). The impedance measurement may be performed by controlling the control unit 30 only within the current/voltage injection measurement unit 10, or the impedance measurement may be performed by controlling the control unit 30 according to a program executed by the measurement calculation unit 50. The results of the impedance measurement are sent to the measurement calculation unit 50. The method for transmitting information to the measurement calculation unit 50 is not particularly limited. Information may be sent from the control unit 30 to the measurement calculation unit 50 through a wire, or may be sent to the measurement calculation unit 50 using another transmission method.

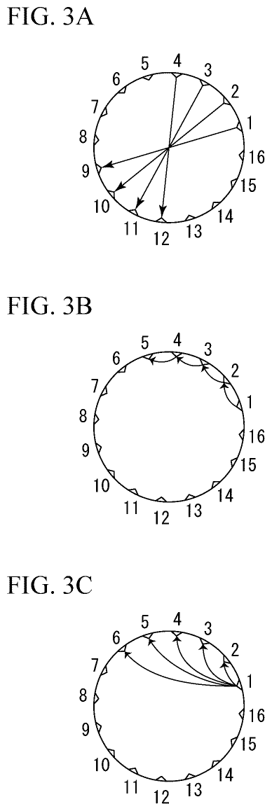

Using the electrode arrangements of FIG. 3A, FIG. 3B, and FIG. 3C as examples, current injection/voltage measurement patterns for the electrodes 21 are explained. For example, the numbers representing the positions of the electrodes 21 are assigned counterclockwise from the first electrode, which acts as a reference. The number M of the current injection/voltage measurement pattern is different for each current injection/voltage measurement pattern. The following description explains the current injection/voltage measurement patterns. Examples of current injection/voltage measurement patterns will be given below, but it should be understood that the present invention is not limited to these patterns.

First, a current injection/voltage measurement pattern using the counter electrode method is described. In this case, a current is injected between a pair of opposing electrodes. For example, explaining with reference to FIG. 3A, current is injected to opposing electrodes, such as the no. 1 electrode and the no. 9 electrode, and the no. 2 electrode and the no. 10 electrode. In the case of FIG. 3A, the number Q of electrodes is 16, thus, there are eight paths in total. The potential difference is measured in electrode pairs, such as the second electrode and third electrode, the third electrode and fourth electrode, excluding the electrode that injects the current and the measurement is carried out from the electrode pair of the second electrode and the third electrode to the electrode pair of the 15th electrode and 16th electrode, thus, there are 13 paths for voltage measurement patterns (potential difference measurement patterns) for one current injection pattern. Accordingly, in the case of the counter electrode method, the number M of measurements (measurement patterns) is 104 paths in total. Here, in a case of injecting a current to measure a potential difference, the measurement pattern becomes a voltage measurement pattern. In a case where a potential difference is applied to measure a current, the measurement pattern is a current measurement pattern.

Next, a current injection/voltage measurement pattern using the adjacent method is described. In this case, a current is injected between adjacent electrodes. For example, explaining with reference to FIG. 3B, current is injected to adjacent electrodes such as the no. 1 electrode and the no. 2 electrode and the no. 2 electrode and the no. 3 electrode. In the case of FIG. 3B, the number Q of electrodes is 16, thus, there are 16 paths in total. The potential difference is measured in pairs of electrodes, such as the third electrode and fourth electrode, excluding the electrode that injects the current and the measurement is carried out from third electrode and the fourth electrode to the 15th electrode and 16th electrode, thus, there are 13 paths for voltage measurement patterns (potential difference measurement patterns) for one current injection pattern. Accordingly, in the case of the adjacent method, the number M of measurements (measurement patterns) is 208 paths in total.

The following description explains a current injection/voltage measurement pattern using the reference method. In this case, potential differences are measured for all combinations between a reference electrode and electrodes other than the reference electrode. For example, referring to FIG. 3C, a current is injected between a reference electrode and electrodes other than the reference electrode, such as the no. 1 electrode and the no. 2 electrode, or the no. 1 electrode and the no. 3 electrode. In the case of FIG. 3C, the number Q of electrodes is 16, thus, there are 16 paths in total. The potential difference is measured in electrode pairs such as the third electrode and fourth electrode excluding the electrode that injects the current and the measurement is carried out from the electrode pair of the third electrode and fourth electrode to the electrode pair of the 15th electrode and 16th electrode, thus, there are 13 paths for voltage measurement patterns (potential difference measurement patterns) for one current injection pattern. Accordingly, in the reference method, the number M of measurements (measurement patterns) is 208 paths in total.

The following description will explain an example in which the in-vivo visualization device 100 of the present embodiment measures a potential difference using the adjacent method. Although this is an example of calculation using one sensor 20, it is also possible to carry out calculations for two or more sensors 20 in the same manner. Furthermore, the in-vivo visualization device 100 according to the present embodiment learns predetermined parameters using sparse Bayesian learning.

### (Image Reconstruction Unit)

The image reconstruction unit 52 of the measurement calculation unit 50 creates an electrical property distribution inside the subject based on predetermined parameters and the potential difference and phase current (may be referred to below as first measurement data), and the current and phase (may be referred to below as second measurement data) measured by the current/voltage injection measurement unit 10. Specifically, the image reconstruction unit 52 creates a multiple measurement matrix from at least one of impedance, resistance, reactance, capacitance, admittance, conductance, susceptance, and phase obtained from the first measurement data or the second measurement data and creates an electrical property distribution from the multiple measurement matrix and predetermined parameters. Here, an example is described in which a multiple measurement matrix is created using impedance. For example, the image reconstruction unit 52 generates a multiple measurement matrix (MMM) of an impedance time difference ΔZ and creates an electrical property distribution inside the subject's body from the created MMM of the impedance time difference ΔZ and the predetermined parameters. The predetermined parameters are, for example, a prior variance vector γ relating to sparsity, a positive definite matrix B relating to temporal correlation, and a variance value λ of a multidimensional normal distribution relating to noise. Initial values of the parameters may be inputted based on data. The initial value may be determined based on, for example, a priori information relating to the electrical properties in a living body, or may be determined empirically by taking into consideration noise or the like in the experiment conditions. The initial electrical property distribution created by the image reconstruction unit 52 based on the initial predetermined parameters is sent to the region of interest identification unit 53. The initial electrical property distribution is sent to the region of interest identification unit 53 to be subjected to an identification process. The post-identification electrical property distribution obtained after the initial electrical property distribution is subjected to the identification process is then sent to the learning unit 54. In a case where the image reconstruction unit 52 creates an updated electrical property distribution based on parameters updated by the learning unit 54, the result is sent to the learning unit 54 or the output unit 55. Specifically, the creation of an updated electrical property distribution in the image reconstruction unit 52 and the updating of the predetermined parameters in the learning unit 54 are repeated until a termination condition described below is satisfied in the learning unit 54. In a case where the updating of the parameters in the learning unit 54 is completed, the image reconstruction unit 52 sends the electrical property distribution to the output unit 55.

The electrical property distribution is not particularly limited and examples thereof include spatial conductivity, spatial permittivity distribution, spatial phase distribution, temporal conductivity distribution, temporal permittivity distribution, temporal phase distribution, conductivity response distribution at an applied frequency, permittivity response distribution at an applied frequency, phase response distribution at an applied frequency, etc. The following description explains a method for creating an electrical property distribution, taking as an example a method for creating a conductivity distribution inside a subject.

The impedance difference ΔZₖ between the impedance between an initial time to and a time tₖ (k=1, 2, . . . , K) obtained from the current or the like measured by the current/voltage injection measurement unit 10 is expressed by Equation (1). Here. J in Equation (1) is a sensitivity matrix expressed by Equation (2). Δσₖ in Equation (1) is the conductivity distribution at the time tₖ expressed by Equation (3). εₖ in Equation (1) is a noise vector expressed by Equation (4). Here, N in Equation (2) and Equation (3) is the total number of meshes (total number of elements) that form the image and M in Equation (2) is the number of measurements (number of measurement patterns). In addition, the symbol formed by combining a/mark with a ∘ mark in Equation (1) indicates the division for each element of a vector.

$$\begin{matrix}[ Equation 1 ] & \\ Δ {Z}_{k} = ( {Z}_{k} - {Z}_{0} ) ∅ {Z}_{0} = J Δ {σ}_{k} + {ε}_{k} & ( 1 )\end{matrix}$$

$$J ∈ {ℝ}^{N \times M} \qquad ( 2 )$$

$${Δσ}_{k} ∈ {ℝ}^{N} \qquad ( 3 )$$

$${ε}_{k} = [ {ε}_{k , 1} , … , {ε}_{k , m} , … , {ε}_{k , M} ] ∈ {ℝ}^{M} \qquad ( 4 )$$

The image reconstruction unit 52 generates ΔZ of the MMM expressed by Equation (5) from ΔZₖ of a column vector. M in Equation (5) is the number of measurements (number of measurement patterns) and K is the number of the time tₖ. ΔZ of the MMM is expressed by Equation (8) from the conductivity distribution Δσ of the MMM expressed by Equation (6) and a noise matrix c of the MMM expressed by Equation (7). When AZ of the MMM in Equation (8) is converted into a column vector, Equation (9) is obtained. Here, * is the symbol for the column vectorization. ΔZ* in Equation (9) is expressed by Equation (10). Δσ* in Equation (9) is expressed by Equation (11). ε* in Equation (9) is expressed by Equation (12). J⁻ in Equation (9) is expressed by Equation (13), in Equation (13) is a unit matrix expressed by Equation (14). In Equation (13), the symbol formed by combining a x mark with a O mark indicates the Kronecker product symbol.

$$\begin{matrix}[ Equation 2 ] & \\ Δ Z = [ Δ {Z}_{1} , … , Δ {Z}_{i} , … , Δ {Z}_{K} ] ∈ {ℝ}^{M \times K} & ( 5 )\end{matrix}$$

$$Δ σ = [ Δ {σ}_{1} , … , {Δσ}_{k} , … , {Δσ}_{K} ] ∈ {ℝ}^{N \times K} \qquad ( 6 )$$

$$ε = [ {ε}_{1} , … , {ε}_{k} , … , {ε}_{K} ] ∈ {ℝ}^{M \times K} \qquad ( 7 )$$

$$Δ Z = J Δσ + ε \qquad ( 8 )$$

$$Δ {Z}^{*} = J Δ {σ}^{*} + {ε}^{*} \qquad ( 9 )$$

$$\begin{matrix}[ Equation 3 ] & \\ Δ {Z}^{*} ∈ {ℝ}^{MK} & ( 10 )\end{matrix}$$

$$Δ {σ}^{*} ∈ {ℝ}^{NK} \qquad ( 11 )$$

$${ε}^{*} ∈ {ℝ}^{MK} \qquad ( 12 )$$

$$\tilde{J} = J ⊗ I ∈ {ℝ}^{MK \times NK} \qquad ( 13 )$$

$$I ∈ {ℝ}^{K \times K} \qquad ( 14 )$$

Here, in sparse Bayesian learning, the prior probability distribution p(Δσ*; Σ^{pre}), likelihood p(ΔZ*|Δσ*; ε*), and posterior probability distribution p(Δσ*|ΔZ*; Σ^{past}) of Δσ are defined. In sparse Bayesian learning using temporal correlation, assuming a multidimensional normal distribution which is a prior covariance matrix Σ^{pre} formed of a zero mean vector 0, a prior variance vector γ relating to sparsity, and a positive definite matrix B relating to temporal correlation, the prior probability distribution p(Δσ*; Σ^{pre}) of Δσ is expressed by Equation (15). The prior variance vector γ is expressed by Equation (16), the positive definite matrix B is expressed by Equation (17) and the prior covariance matrix Σ^{pre} is expressed by Equation (18A) and Equation (18B). It is possible to adjust the locality by changing the size of the prior variance vector γ relating to sparsity. In Equation (18B), when the positive definite matrix B is set to be common to all elements N, it is possible to prevent overfitting of the learning, which is preferable. Overfitting refers to undesirable machine learning behavior that occurs when a machine learning model provides accurate predictions on training data, but not on new data.

$$\begin{matrix}[ Equation 4 ] & \\ p ( {Δσ}^{*} ; {Σ}^{pre} ) \sim N ( 0 , {Σ}^{pre} ) & ( 15 )\end{matrix}$$

$$γ = [ {γ}_{1} , … , {γ}_{n} , … , {γ}_{N} ] ∈ {ℝ}^{N} \qquad ( 16 )$$

$$B ∈ {ℝ}^{k \times k} \qquad ( 17 )$$

$${Σ}^{pre} ∈ {ℝ}^{NK \times NK} \qquad ( 18 A )$$

$${Σ}^{pre} = [ \begin{matrix}{γ}_{1} B & & 0 \\ & ⋱ & \\ 0 & & {γ}_{N} B\end{matrix} ] \qquad ( 18 B )$$

Assuming that the noise probability distribution p(ε*) is a multidimensional normal distribution with a zero mean vector 0 and an independent and identical noise covariance matrix λI for each element, the result is expressed by Equation (19). Here, the noise covariance matrix λI is expressed by Equation (20). Here, λ in Equation (20) is the variance value of the multidimensional normal distribution. From Equation (9) and Equation (19), the likelihood p(ΔZ*|Δσ*; ε*) of the impedance time difference ΔZ* is expressed by Equation (21) as a conditional probability distribution.

$$\begin{matrix}[ Equation 5 ] & \\ p ( {ε}^{*} ) \sim N ( 0 , λ I ) & ( 19 )\end{matrix}$$

$$λ I ∈ {ℝ}^{MK \times MK} \qquad ( 20 )$$

$$p ( Δ {Z}^{*} \vert Δ {σ}^{*} ; {ε}^{*} ) \sim N ( \tilde{J} {Δσ}^{*} , λ I ) \qquad ( 21 )$$

Bayes' theorem, that is, the relationship between the prior probability distribution Equation (19) and the likelihood Equation (21), is as in Equation (22). According to Equation (22), it is also possible to assume that the posterior probability distribution is also a normal distribution, thus, the posterior probability distribution is expressed by Equation (23) from the mean vector of conductivity and a posterior covariance matrix Σ^{post}. The mean vector of conductivity is expressed by Equation (24) and the posterior covariance matrix Σ^{post} is expressed by Equation (25). Since the normal distribution has a maximum probability at the expected value, a maximum a posteriori probability estimation solution (MAP estimation solution) for Δσ is the mean vector of conductivity in Equation (23) and is expressed by Equation (26) and Equation (27). Here, T is a transpose symbol. The conductivity distribution Δσ can be obtained by converting Δσ* in Equation (26) into Δσ of N×K-dimensions. Thus, it is possible to create an electrical property distribution (here, conductivity distribution) inside the subject's body.

$$\begin{matrix}[ Equation 6 ] & \\ p ( {Δσ}^{*} \vert Δ {Z}^{*} ; {Σ}^{post} ) ∝ p ( Δ {Z}^{*} \vert Δσ ; {ε}^{*} ) p ( {Δσ}^{*} ; {Σ}^{pre} ) & ( 22 )\end{matrix}$$

$$p ( {Δσ}^{*} \vert Δ {Z}^{*} ; {Σ}^{post} ) \sim N ( \overline{{Δσ}^{*}} , {Σ}^{post} ) \qquad ( 23 )$$

$$\overline{{σ}^{*}} ∈ {ℝ}^{NK} \qquad ( 24 )$$

$${Σ}^{post} ∈ {ℝ}^{NK \times NK} \qquad ( 25 )$$

$${Δσ}^{*} = {Δσ}^{*} = \frac{1}{λ} {Σ}^{post} {\tilde{J}}^{T} Δ {Z}^{*} = {[ {λ ( {Σ}^{pre} )}^{- 1} + {\tilde{J}}^{T} \tilde{J} ]}^{- 1} {\tilde{J}}^{T} Δ {Z}^{*} \qquad ( 26 )$$

$$∵ {Σ}^{post} = {[ {( {Σ}^{pre} )}^{- 1} + \frac{1}{λ} {\tilde{J}}^{T} \tilde{J} ]}^{- 1} \qquad ( 27 )$$

### (Region of Interest Identification Unit 53)

The region of interest identification unit 53 performs a predetermined process on the initial electrical property distribution obtained by the image reconstruction unit 52 and identifies a region of interest. The following describes a method for identifying a region of interest using a predetermined process.

The region of interest identification unit 53 performs preprocessing on the electrical property distribution obtained by the image reconstruction unit 52 and creates a region of interest index distribution. The preprocessing is not particularly limited as long as it is possible to identify the biological tissue that is the region of interest and examples thereof include standard deviation processing, difference processing between the maximum value and minimum value at a predetermined pixel a (max(a)-min(a)), normalized difference processing for changes in the maximum value and minimum value ((max(a)·min(a))/min(a)), first-order differential processing (∂y/∂k: y is the target electrical property), second-order differential processing (∂²y/∂k²), various types of frequency analysis such as a Fourier transform or a wavelet transform, etc. For the above, various types of matrix processing (singular value decomposition, main component analysis, independent component analysis, etc) are performed based on the total number of pixels N and the total number of frames K, using an N×K matrix. In the preprocessing described above, temporal processing may be performed in a case where there is a desire to observe spatial local changes and spatial processing may be performed in a case of observing temporal local changes. The following description explains an example of standard deviation processing for observing spatial changes.

Using the conductivity distribution obtained by the image reconstruction unit 52, a region of interest index distribution (here, a temporal standard deviation image v) expressed by Equation (28) is calculated as in Equation (29). Here, Δσ_{k,n} in Equation (29) is the value of the conductivity of the n-th element at the time tₖ and <σₙ> is the time average of the value of the n-th element.

In a field (here, a field that changes over time) where the background electrical properties (here, conductivity) undergo significant changes temporally or spatially, in order to extract local changes in time and space in the electrical properties within the region of interest (RO), elements other than the ROI are removed and, in order to identify the elements of the ROI, an identification process is performed based on the region of interest index distribution (here, the temporal standard deviation image v) and a predetermined threshold value c and a post-identification electrical property distribution is created. The identification process is not particularly limited as long as it is possible to remove elements other than the ROI and may be, for example, a process of removing an element in a case where it is determined whether each value of the region of interest index distribution falls within the range of the threshold and the values do not fall within the range of the threshold. For example, processing is performed as in Equation (30) and the post-identification electrical property distribution obtained from Equation (30) is converted into a column vector again and sent to the learning unit 54.

Here, the predetermined threshold value c is determined from the magnitude relationship between the region of interest ROI and a of the background based on a priori information relating to a for each biological tissue. For example, the predetermined threshold value c may be determined according to the ratio of the number of pixels in the region of interest (ROT) to the total number of pixels of the image.

$$\begin{matrix}[ Eqaution 7 ] & \\ {ν = {ν}_{1} , … , {ν}_{n} , … , {ν}_{N} ]}^{T} ∈ {ℝ}^{N} & ( 28 )\end{matrix}$$

$${ν}_{n} = \sqrt{\frac{1}{K} {\sum }_{k = 1}^{K} {( {Δσ}_{k , n} - 〈 {Δσ}_{n} 〉 )}^{2}} \qquad ( 29 )$$

$${Δσ}_{k , n} = \{ \begin{matrix}{Δσ}_{k , n} , & in the case of {ν}_{n} < c \\ 0 , & in the case of {ν}_{n} \geq c\end{matrix} \qquad ( 30 )$$

The learning unit 54 updates predetermined parameters based on the updated electrical property distribution obtained by the image reconstruction unit 52 or the post-identification electrical property distribution obtained by the region of interest identification unit 53. Specifically, the learning unit 54 learns a parameter matrix Θ (for example, λ, γ, B) by maximizing a negative log marginal likelihood-log p (ΔZ*|Θ) or by maximizing an average data likelihood Q(Θ). For example, in a case where each probability distribution is assumed to be a normal distribution, a likelihood function L(Θ) is acquired by considering only the logarithm of the marginal likelihood.

Here, the likelihood function L(Θ) is expressed by Equation (31). Σ^{likelihood} in Equation (31) is expressed by Equation (32). The log marginal likelihood-log p (ΔZ*|Θ) may be maximized by maximizing L(O). In the present embodiment, a method for learning using the average data likelihood Q(O) will be explained, but the invention shall not be limited to the following method. Specifically, the following description explains a case in which an expectation-maximization (EM) algorithm is used to maximize the average data likelihood Q(Θ). The average data likelihood is expressed by Equation (33). E[⋅] in Equation (33) represents the expected value. The updated value of the EM algorithm, that is, the Θ that maximizes the average data likelihood Q(Θ) is the Θ for which the partial differential of Q(Θ) with respect to Θ is zero.

The following description explains the updating of γ and B in the parameter matrix Θ. By ignoring the first term on the right-hand side regarding λ in Equation (33), Equation (34) is obtained. Here, Γ in Equation (34) is a diagonal matrix having γ as diagonal elements, which is expressed by Equation (35). Trace{ } is a square matrix trace. The updated value of γₙ, which is the n-th element of γ, is the γₙ for which the partial differential of Equation (34) with respect to γₙ is zero and is updated as in Equation (36) using an iterative calculation number iter. Here, Δσ*ₙ in Equation (36) is expressed by Equation (37) and represents temporal change of the n-th element. Σ^{post}ₙ in Equation (36) is expressed by Equation (38) and represents the corresponding covariance matrix.

Similarly, the updated value of B is the B for which the partial differential of Equation (34) with respect to B is zero and is updated as in Equation (39).

The following description explains the updating of λ within Θ. By ignoring the second term on the right-hand side regarding γ and B in Equation (33), the updating is expressed by Equation (40). The updated value of λ is the λ for which the partial differential of Equation (40) with respect to λ is zero and is updated as in Equation (41). The Θ learning procedure is repeated until a termination condition is satisfied. For example, the termination condition, for example, may be set to be satisfying either: an error δ expressed by Equation (42) being a minimum value δ min, or iter being a maximum number of iterations itermax.

$$\begin{matrix}[ Equation 8 ] & \\ L ( Θ ) = log \vert {Σ}^{likelihood} \vert + {( Δ {Z}^{*} )}^{T} {( {Σ}^{likelihood} )}^{- 1} Δ {Z}^{*} & ( 31 )\end{matrix}$$

$${Σ}^{likelihood} = λ I + \tilde{J} {Σ}^{pre} {\tilde{J}}^{T} ∈ {ℝ}^{MK \times MK} \qquad ( 32 )$$

$$Q ( Θ ) = E [ log p ( Δ {Z}^{*} \vert {Δσ}^{*} ; λ ) ] + E [ log p ( Δ {σ}^{*} ; γ , B ) ] \qquad ( 33 )$$

$$Q ( γ , B ) ∝ - \frac{K}{2} ( \vert Γ \vert ) - \frac{N}{2} log ( \vert B \vert ) - \frac{1}{2} Trace \{ ( {Γ}^{1} ⊗ {B}^{1} ) [ {Σ}^{post} + {{Δσ}^{*} ( {Δσ}^{*} )}^{T} ] \} \qquad ( 34 )$$

$$Γ ∈ {ℝ}^{N \times N} \qquad ( 35 )$$

$$\begin{matrix}[ Equation 9 ] & \\ {γ}_{n}^{iter 1} = \frac{1}{K} Trace \{ {( {B}^{iter} )}^{1} [ {( {Σ}_{n}^{post} )}^{iter} + {{Δσ}_{n}^{* iter} ( {Δσ}_{iter}^{*} )}^{T} ] \} & ( 36 )\end{matrix}$$

$${Δσ}_{n}^{*} = {Δσ}^{*} ( ( n - 1 ) K + 1 : nK ) \qquad ( 37 )$$

$${Σ}_{n}^{post} = {Σ}^{post} ( ( n - 1 ) K + 1 : nK , ( n - 1 ) K + 1 : nK ) \qquad ( 38 )$$

$${B}^{iter + 1} = \frac{1}{N} {\sum }_{n = 1}^{N} \frac{{{( {Σ}_{n}^{post} )}^{iter} + {Δσ}_{n}^{* iter} {Δσ}_{n}^{* iter} )}^{T}}{{γ}_{n}^{iter}} \qquad ( 39 )$$

$$Q ( λ ) ∝ - \frac{MK}{2} log λ - \frac{1}{2 λ} E [ {‖ Δ {Z}^{*} - \tilde{J} {Δσ}^{*} ‖}_{2}^{2} ] \qquad ( 40 )$$

$${λ}^{iter + 1} = \frac{1}{MK} \{ {‖ Δ {Z}^{*} - \tilde{J} {Δσ}^{* iter} ‖}_{2}^{2} + {λ}^{iter} [ NK - Trace ( {Σ}^{post} {( {Σ}^{pre} )}^{1} ) ] \} \qquad ( 41 )$$

$$δ = {‖ {Δσ}^{* iter + 1} - {Δσ}^{* iter} ‖}_{2} / {‖ {Δσ}^{* iter + 1} ‖}_{2} \qquad ( 42 )$$

### (Output Unit)

The output unit 55 outputs the updated electrical property distribution obtained by the image reconstruction unit 52. The output destination of the updated electrical property distribution is not particularly limited. For example, the output unit 55 may output and store the updated electrical property distribution in a storage device such as an HDD inside the in-vivo visualization device 100. In addition, the output unit 55 may output the updated electrical property distribution to a display device such as an external liquid crystal display.

### (In-Vivo Visualization Method)

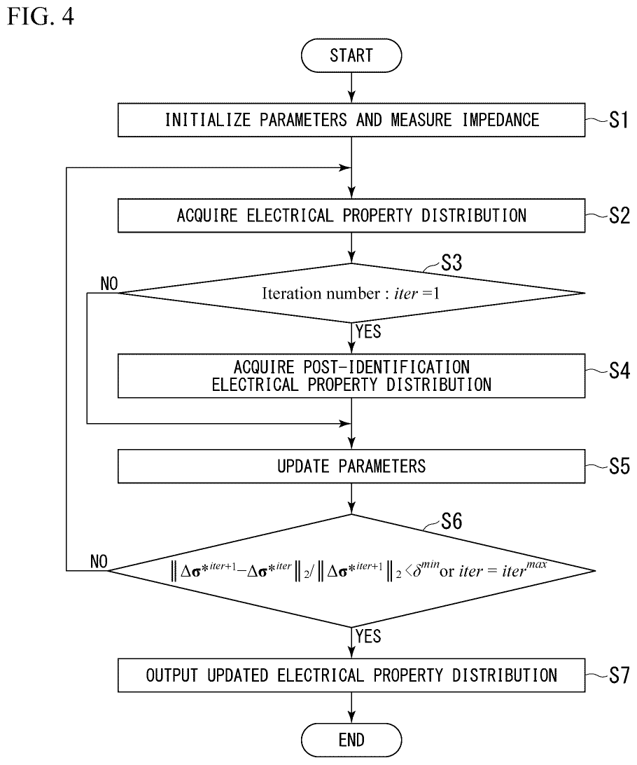

Next, an in-vivo visualization method using the in-vivo visualization device 100 according to the present embodiment is described. FIG. 4 is a flowchart of the in-vivo visualization method according to the present embodiment.

In the in-vivo visualization method, predetermined parameters (here, γ, B, and λ) are initialized and impedance measurement (measurement of the ratio of the potential difference to the current and the phase thereof) is performed using the current/voltage injection measurement unit 10 (S1). Thereafter, an electrical property distribution is created based on the initialized predetermined parameters and the measured impedance (S2). Next, it is determined whether the distribution is the initial electrical property distribution created based on the initialized predetermined parameters or the updated electrical property distribution created based on the predetermined parameters updated by the learning unit 54 (S3). In such a case, a case of iter=1 is determined to be the initial electrical property distribution. In the case of the initial electrical property distribution, the region of interest identification unit 53 performs an identification process to identify the region of interest on the initial electrical property distribution and creates a post-identification electrical property distribution (S4). Next, the learning unit 54 updates the predetermined parameters based on the post-identification electrical property distribution or the updated electrical property distribution (S5). Next, it is determined whether the termination condition (whether the error S is the minimum value or whether a predetermined number of iterations have been carried out) is satisfied (S6). In a case where the termination condition is satisfied, the output unit 55 outputs the updated electrical property distribution (S7). In a case where the termination condition is not satisfied, the process returns to S2.

A detailed description of the in-vivo visualization device 100 is as given above. The in-vivo visualization device 100 according to the present embodiment is able to identify local changes in time and space.

The technical scope of the present invention is not limited to the above embodiments and it is possible to make various changes thereto in a range not departing from the spirit of the present invention.

In addition, it is possible to appropriately replace the constituent elements in the embodiments with well-known constituent elements in a range not departing from the spirit of the present invention, and such modifications may be combined as appropriate.

### EXAMPLES

The present invention is further explained by the following Examples, but the present invention should not be considered to be limited to these Examples.

### Experiment Example 1

The following description describes an example of an experiment conducted to verify the effectiveness of the in-vivo visualization device 100 according to the present embodiment.

Using numerical simulation under conditions simulating lymphedema using the in-vivo visualization device of the present invention, for a field of a background of SAT and muscle in which there are significant temporal changes in conductivity, it was confirmed whether or not it was possible to extract temporal and spatial local changes in a in the vicinity of veins, which are characteristic of lymphedema, and the precision of the simulation was qualitatively examined. Furthermore, the conductivity ratios ρ^{GSV/SAT} and ρ^{GSV/muscle} were examined quantitatively as parameters.

As the Prior Art Example, image reconstruction was performed using the iterative Gauss-Newton method, which is a conventional method. The equation used for the image reconstruction is Equation (43). Δσ^{iter}ₖ in Equation (43) indicates the conductivity distribution at the time tₖ in the iter-th iteration. I in Equation (43) is a unit matrix expressed by Equation (44) and η is a regularization parameter. J is a sensitivity matrix and J^{T} is a transposed matrix of a sensitivity matrix T. ΔZₖ in Equation (43) is the measurement impedance. A measurement impedance ΔZₖ was calculated by the finite element method (FEM) using MATLAB (registered trademark) R2020a (Mathworks, Natick, MA). The number of elements for quasi-problem calculation was 2946 and the number of elements N for the inverse problem calculation was N=4560.

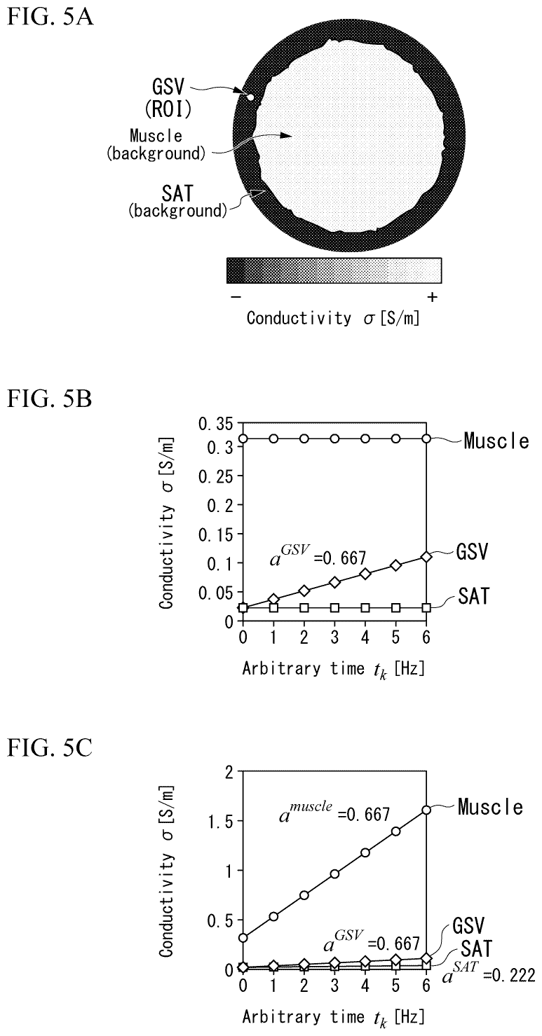

FIG. 5A is a simple simulated image of a human calf. The simulated image is composed of three parts: the great saphenous vein (GSV), subcutaneous adipose tissue (SAT), and muscle. The ROI was the GSV.

In order to simulate the physiological phenomena of two types of edema progression, the conductivity value σ of each site was changed over time from t₀ to t₆. FIG. 5B shows the lymphedema simulating conditions of Condition 1, in which only a of the GSV changes over time at a constant rate of change a^{GSV} and a of the SAT and the muscle do not change over time. FIG. 5C shows the venous edema simulating condition of Condition 2, in which σ of the GSV and the muscle change over time at a constant rate of change a^{GVS}=a^{muscle}(=0.667) and σ of the SAT changes over time at a rate of change a^{SAT} (=0.222<a^{GSV}).

The σ at the initial time to for the SAT and muscle in the two conditions was set to 0.022 [S/m] for a of SAT and 0.321 [S/m] for a of muscle, and a of GSV at to (=0.022 [S/m]) used the same value as for the SAT. In general, σ during edema is proportional to the amount of water, thus, the rate of change a^{GSV} was calculated as a^{GSV}=0.667 from the change in water content in a calf in a standing state for 60 minutes. Furthermore, since SAT changes more slowly than GSV, a^{SAT}=a^{GSV}/3.

The sensitivity matrix J was calculated under homogeneous conditions, that is, using σ=1.0 [S/mn]. The injected current I₀ and the applied frequency f were set as I₀=1 mA and f=1 kHz, respectively, and the adjacent method was applied to the applied pattern.

The threshold value c in Equation (30) was set to the median value of a temporal standard deviation image v, for the initial values of λ, γ, and B, λ was set to 0.01, γ was set as in Equation (45), and B was set as in Equation (46). In addition, Gaussian noise was added to Z such that the SN ratio was 40 dB. In the two conditions, the maximum number of iterations iter^{max}=10 was set in the Example (image reconstruction unit 52+region of interest identification unit 53+learning unit 54: with ROI identification), the Comparative Example (image reconstruction unit 52+learning unit 54: without ROI identification), and the iterative Gauss-Newton method (Equation (43)), which is a conventional method.

The regularization parameter η in the iterative Gauss-Newton method was set to η=4.17-10⁻⁶ (lymphedema simulating condition) and η=1.80×10⁻⁵ (venous edema simulating condition) using P. C. Hansen's L-curve method.

$$\begin{matrix}[ Equation 10 ] & \\ {Δ}_{k}^{iter 1} = {Δσ}_{k}^{iter} + {( {J}^{T} J + η \cdot I )}^{- 1} {J}^{T} ( Δ {Z}_{k} - J {Δσ}_{k}^{iter} ) & ( 43 )\end{matrix}$$

$$I ∈ {ℝ}^{N \times N} \qquad ( 44 )$$

$$γ = {[ 1 , … , 1 , … , 1 ]}^{T} ∈ {ℝ}^{N} \qquad ( 45 )$$

$$B = I ∈ {ℝ}^{K \times K} \qquad ( 46 )$$

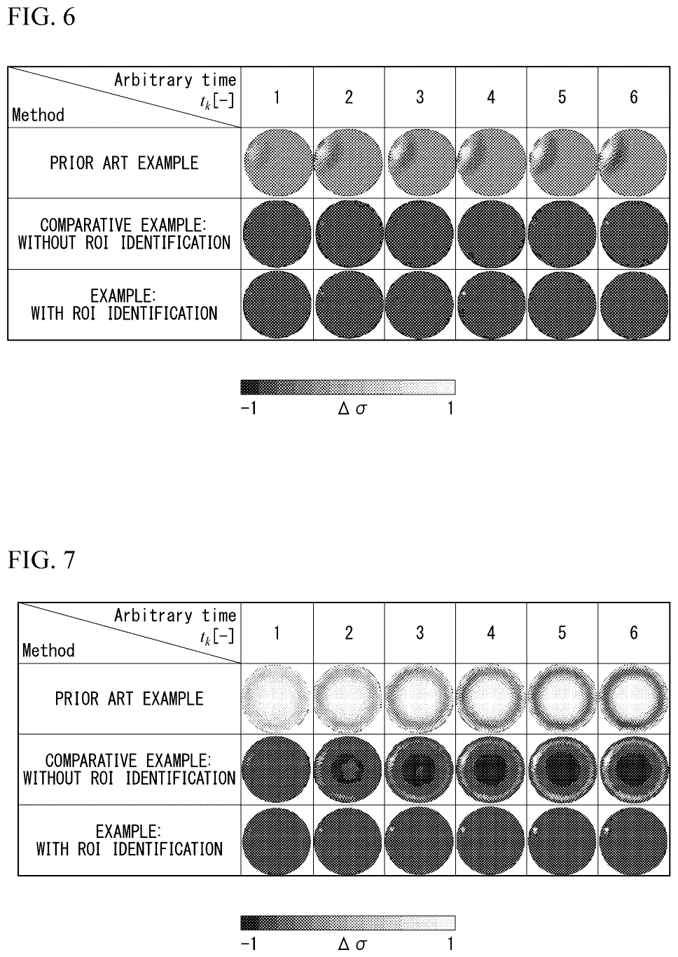

FIG. 6 shows the conductivity distribution Δσ which was image-reconstructed under Condition 1 using the Example (image reconstruction unit 52+region of interest identification unit 53+learning unit 54: with ROI identification), the Comparative Example (image reconstruction unit 52+learning unit 54: without ROI identification), and the Prior Art Example (Gauss-Newton method). Under Condition 1, using the iterative Gauss-Newton method, it was possible to image the temporal change in conductivity in the vicinity of the GSV, but the image was blurred compared to the original image. Furthermore, the influence of ringing artifacts was also significant. On the other hand, the Example and Comparative Example using sparse Bayesian learning were able to extract only local conductivity changes.

Next, FIG. 7 shows the conductivity distribution Δσ which was image-reconstructed under Condition 2 using the Example (image reconstruction unit 52+region of interest identification unit 53+learning unit 54: with ROI identification), the Comparative Example (image reconstruction unit 52+learning unit 54: without ROI identification), and the Prior Art Example (Gauss-Newton method). Under Condition 2, the iterative Gauss-Newton method was able to image the boundary between the SAT and the muscle, but was not able to image the temporal change in conductivity in the vicinity of the GSV. This is considered to be because it was not possible to make the background a constant condition between the initial time to and the time tₖ due to all sites undergoing temporal changes. In contrast, the Comparative Example (image reconstruction unit 52+learning unit 54: without ROI identification) was able to image the conductivity change in the SAT and also extract the vicinity of the GSV having particularly significant temporal changes in that SAT. However, circular artifacts were generated due to the influence of the changes in the SAT. In the case of the Example, it was possible to extract only the conductivity change in the vicinity of the GSV. Despite all of the sites undergoing temporal changes, performing the region of interest identification process made it possible to clearly extract the conductivity change in the vicinity of the GSV, where the temporal and spatial local changes were particularly significant in the region of interest ROL. In the present simulation result, it is clear that the in-vivo visualization device 100 is capable of visualizing and measuring local a which undergoes temporal change.

### Experiment Example 2

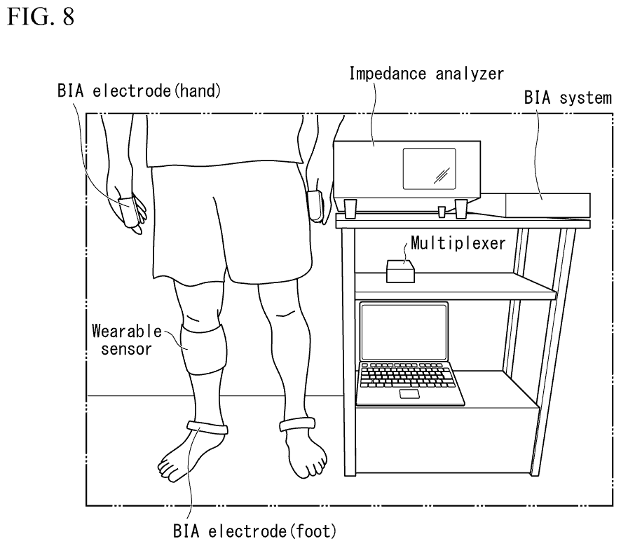

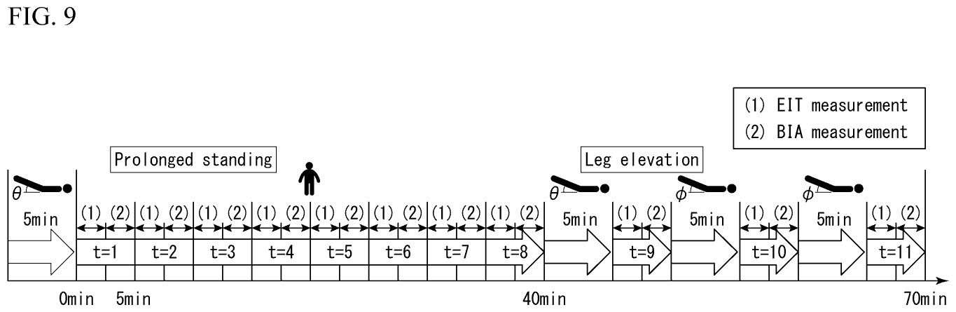

Using the in-vivo visualization device 100, in-vivo visualization was carried out for 15 healthy subjects. A schematic diagram of the measurement device used in Experiment Example 2 is shown in FIG. 8. As shown in FIG. 8, sensors for the conventional bioelectrical impedance analysis were also attached to the hands and feet of the subjects, and these conventional sensors were connected to the bioelectrical impedance analyzer.

The in-vivo visualization device according to the present embodiment is formed of a sensor formed of 16 dry electrodes (5×10 mm) attached to a position on the right calf, a digital multiplexer connected to the sensor, an impedance analyzer (IM3570, HIOKI, Japan), and a control PC. Using a coaxial cable, an impedance analyzer connected to a digital multiplexer, and a PC including edema identification method software, the impedance Z measured by the impedance analyzer is transmitted to the PC using a USB cable. A conventional BIA device is formed of electrodes (two for the hands and two for the feet) (InBody S10, InBody Japan Inc.). It was confirmed in preliminary tests that the measurements of the in-vivo visualization device according to the present embodiment and the conventional BIA device do not interfere with each other, even if the electrodes are attached, as long as both measurements are not performed at the same time.

FIG. 9 shows the experimental protocol relating to prolonged standing and leg elevation to induce local spatiotemporal changes (LSTC) in extracellular fluid (ECF) of the subcutaneous adipose tissue (SAT). At the beginning of the experiment, subjects lay horizontally on a reclining bed and performed leg raises at an angle φ=30 degrees to reduce leg swelling influenced by previous activities such as exercise and muscle fatigue. During the prolonged standing, the subjects stood for 40 minutes without making small movements to induce an increase in ECF volume and measurements by the in-vivo visualization device and measurements by the conventional BIA device were performed continuously, taking 3 minutes and 2 minutes respectively. The above-described process was repeated 8 times (k=1-8: tₖ=5/10/15/20/25/30/35/40 min) for each subject. During the leg elevation, the subjects used a reclining bed to raise their legs for 3 minutes and then slowly stood up. Thereafter, the measurements by the in-vivo visualization device and measurements by the conventional BIA device were performed continuously for a total of 8 minutes to induce ECF volume reduction. The above-described process was repeated three times for each subject (k=8-11: tₖ=50/60/70 min).

In Experiment Example 2, 15 healthy subjects participated (9 males, 6 females, age: 23.1±2.3 years, body mass index: 23.1±4.9 kg/m²). Impedance Z was measured in various time frames (k=1-11) using a current 10=1 mA at two frequencies f1=1.6 kHz and f2=4.7 kHz, selected to account for current pathways penetrating only the extracellular space. Next, the impedance Z was used to carry out image reconstruction of the frequency difference conductivity distribution Δσ using a finite element method (FEM) mesh generated by NETGEN supported by MATLAB (registered trademark) R2020a (Mathworks). To quantify local spatiotemporal changes (LSTC) to evaluate lower limb edema, spatial-mean conductivity <Δσ>SAT was calculated using the separated SAT.

For comparison, the conventional BIA was measured on the right leg at a frequency f=5 kHz and the ECF was determined. These two parameters were compared using the correlation coefficient R, for which the t-test significance level was set at 0.05. Furthermore, the normalized values with mean 0 and standard deviation 1 of the measurements using the in-vivo visualization device and measurements using the conventional BIA, that is, the normalized spatial-mean conductivity (<Δσ>^{SAT}) and the normalized conventional impedance Z^{BIA} were calculated and the trends thereof were evaluated.

To acquire the position of the great saphenous vein (GSV) in the LSTC, ultrasound images were acquired using a high-frequency matrix probe (LOGIQePremium, GE Healthcare, Japan) with respect to the positions of the electrodes of the sensor of the present disclosure. The minimum error bound and maximum number of iterations of the in-vivo visualization device were optimized by being set to δmin=1×10⁻² and iter^{max}=15, respectively. The following describes the influence of these parameters.

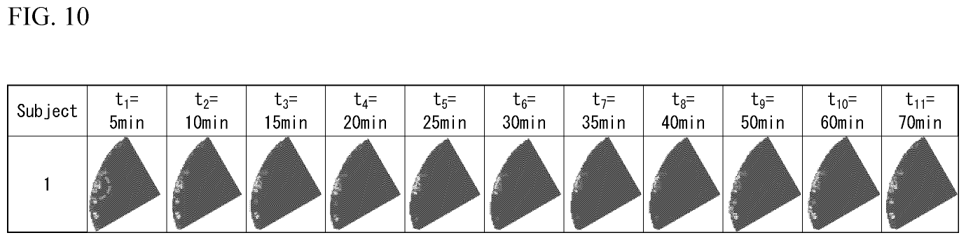

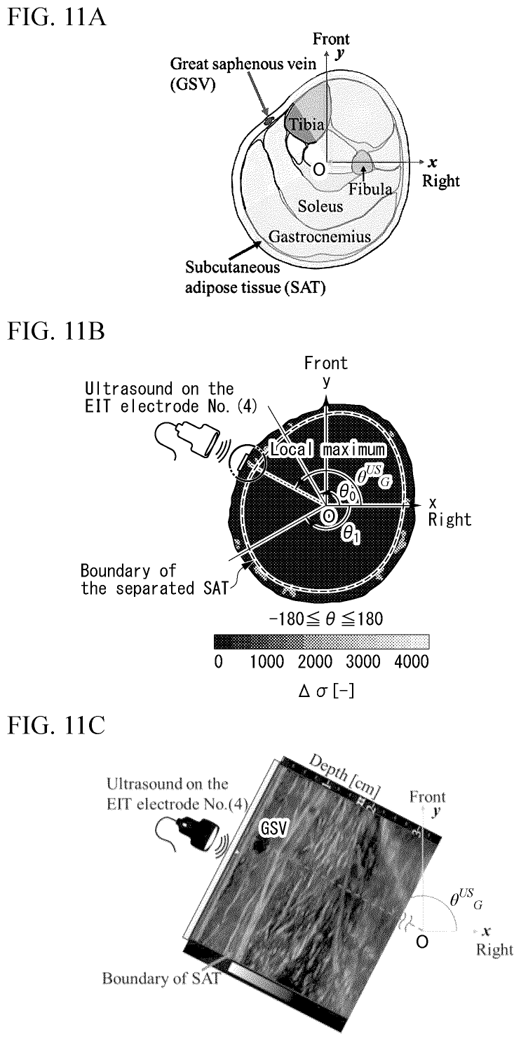

The regularization parameter β for temporal correlation was set to β=4. The threshold value c was set to 25% of the time standard deviation image v. FIG. 10 shows the frequency difference conductivity distribution Δσ of a certain subject, which was reconstructed by the in-vivo visualization device. Trimming was carried out for the calf angle Δσ=120° and θ₁=−150° of a certain subject over a prolonged period (shown in FIG. 11B). The dimensions of the human calf are based on an x-y-z Cartesian coordinate system in which the x axis and y axis are perpendicular to the z axis and the z axis is along the direction of gravity. The dashed-line circle indicates the local maximum position of Δσ relating to the position of the great saphenous vein (GSV). Since the local spatiotemporal changes (LSTC) associated with the GSV are small and difficult to distinguish in the entire image, the entire circular image is only shown in a representative example in FIG. 11B and Δσ was trimmed for all 15 subjects. The angles θ₀ and θ₁ in FIG. 10 are selected to emphasize the Δσ in the vicinity of the GSV position positioned on the anteromedial side of the tibia. Although there was some variation among the subjects, the EIT electrode numbers where the GSV was observed in ultrasound images were positioned near EIT electrode numbers (3) to (5). Accordingly, it is possible to accurately monitor the LSTC of the extracellular fluid (ECF) of separated subcutaneous adipose tissue (SAT) using Au. That is, LSTC decreases during prolonged standing and LSTC increases during leg elevation. High Δσ is also observed at locations other than near the GSV. This is considered to be because the ECF is retained by capillaries near comparatively large veins. More interestingly, locations close to the GSV exhibited the maximum Δσ for almost all of the subjects.

FIG. 11A is a cross-section of a typical human calf, FIG. 1B is the Δσ of subject no. 12, and FIG. 11C is an ultrasound image in the vicinity of the EIT electrode no. (4) of the subject. In FIG. 11B, the white dashed line indicates the boundary of the separated SAT. In contrast, the dashed line in FIG. 11C indicates the direction from the origin of the ultrasound image to the GSV position and the direction from the origin to the maximum value position, with an angle θ^{USG}=150° on the dashed line being the same at an angle θ^{EITG}=150°. From these figures, it was confirmed that the LSTC position of Δσ was indicated in the separated SAT rather than in muscle compartments or bone. In particular, it was confirmed that it was possible to associate the maximum position of Δσ, shown as a dashed line circle in FIG. 11B, with the GSV position.

The following description explains the reason why the maximum value of Δσ (dashed line circle in FIG. 10) is associated with the position of the great saphenous vein (GSV). The global minimum value of a sparse Bayesian learning framework is always the sparsest, due to the sparsity-related prior variance vector γ controlling the individual elements of Δσ. The GSV is positioned between the superficial fascia and the saphenous fascia and the depth position thereof depends on the thickness of the subcutaneous tissue. Furthermore, the GSV plays an important role in returning blood from the subcutaneous tissue of the legs to the heart. Several large maximum values were observed in FIG. 10, which are presumed to be related to ECF diffused from capillaries near the GSV. From the above, it was understood that it is possible for the in-vivo visualization device to extract the temporal correlation of the Δσ of the separated SAT.

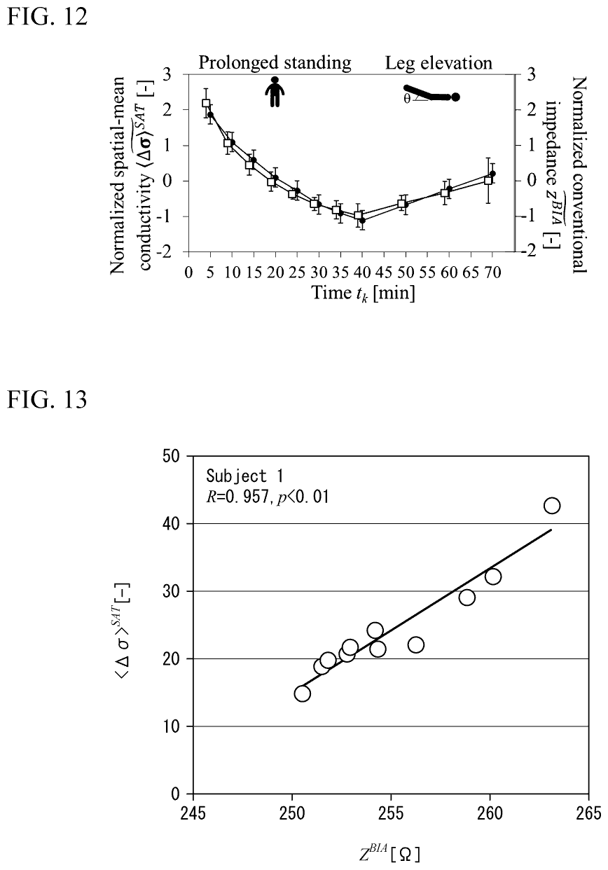

FIG. 12 shows the normalized spatial-mean conductivity <Δσ>^{SAT} in the separated SAT and the normalized conventional BIA impedance z^{RIA} during prolonged standing and leg elevation. In FIG. 12, <Δσ>^{SAT} and z^{BIA} decrease with prolonged standing and <Δσ>^{SAT} and z^{BIA} increase with lower limb elevation. Although the standard deviation of <Δσ>^{SAT} was high relative to the standard deviation of z^{BIA}, <Δσ>^{SAT} showed the ECF trend well.

FIG. 13 shows the correlation between the spatial-mean conductivity <Δσ>^{SAT} of the separated SAT and the conventional impedance z^{BIA} for the calf of a certain subject. The vertical axis is the spatial-mean conductivity <Δσ>^{SAT} and the horizontal axis is z^{BIA}[Ω]. The correlation coefficient R and p value between the two parameters are also shown. As shown in FIG. 13, there was a strong positive correlation between Δσ and z^{BIA}, and this tendency was the same for all subjects. The score was 0.715<R<0.957 (p<0.05). Accordingly, it was confirmed that the LSTC exhibited a similar trend to the temporal changes of zBIA and that it was possible to visualize LSTC not possible with the conventional BIA.

Regarding the range of each value, zBIA was in the range of 170 to 340[Ω], whereas <Δσ>^{SAT} was in the range of 10 to 1400 [−]. The conventional BIA primarily evaluates static conditions based on absolute values, while in-vivo visualization devices primarily evaluate dynamic trends based on relative values. The variation in <Δσ>^{SAT} was larger than the variation in z^{BIA}, with a maximum difference of approximately 2 orders of magnitude.

The following description explains the reason for the occurrence of local spatiotemporal changes (LSTC) in the frequency difference conductivity distribution Δσ in separated subcutaneous adipose tissue (SAT). During prolonged standing, the gravity of the blood increases hydrostatic pressure in the lower limbs and increases capillary filtration into the extracellular space of tissues such as SAT. At the same time, reabsorption in the extracellular fluid (ECF) is decreased due to an increase in the osmotic pressure of the extracellular space, which mainly depends on the sodium ion concentration. Accordingly, the proportional relationship of the ratio values shown in Equation (47) is derived. Here, L^{ECF} and L^{ICF} are the volume of the ECF and the volume of intracellular fluid (ICF), respectively, and Na^{+ECF} and Na^{+ICF} are the sodium ion concentration of ECF and the sodium ion concentration of ICF, respectively. The conductivity distribution σ of an NaCl solution, which is the main component of an ECF, is frequency-independent at frequencies f<1 MHz, but the conductivity distribution σ of the protein solution, which is a subcomponent of the ECF, is frequency-dependent at low frequencies. The impedance Z measured at low frequencies of less than f=5 kHz represents the ECF. Since the protein concentration in healthy people remains constant during prolonged standing, the normalized spatial-mean conductivity <Δσ>^{SAT} in FIG. 12 is associated with a decrease in the frequency dependence of a in the ECF caused by an increase in Na⁺.

From the above, in the case of a healthy person, the relationship of Equation (48) is derived. According to Equation (48), the volume change ΔL^{ECF} of the ECF of the right leg during prolonged standing and leg elevation is estimated from the spatial-mean conductivity <Δσ>^{SAT} as shown in Equation (49). Here, ΔL^{ECF} and <Δσ>^{SAT} in the kth time frame are considered. w is the subject's body weight, ρ^{TBF} is the ratio (here 0.6) of total body fluid (TBF) to total body weight, ρ^{TBF/ECF} is the ratio (here 0.29) of ECF to TBF, and ρ^{RL} is the ratio (here, 0.1) of the right leg to the whole body.

$$\begin{matrix}[ Equation 11 ] & \\ \frac{{L}^{HCF}}{{L}^{ICF}} ∝ \frac{{Na}^{+ HCF}}{{Na}^{+ ICF}} & ( 47 )\end{matrix}$$

$$\frac{1}{{〈 Δσ 〉}^{SAT}} ∝ \frac{{L}^{ECF}}{{L}^{ICF}} ∝ \frac{{Na}^{+ HCF}}{{Na}^{+ ICF}} \qquad ( 48 )$$

$$Δ {L}_{k}^{ECF} = \vert \frac{{〈 Δσ 〉}_{k}^{SAT} - {〈 Δσ 〉}_{1}^{SAT}}{{〈 Δσ 〉}_{1}^{SAT}} \vert \times w \times {ρ}^{TBF} \times {ρ}^{ECF / TBF} \times {ρ}^{RL} [ l ] \qquad ( 49 )$$

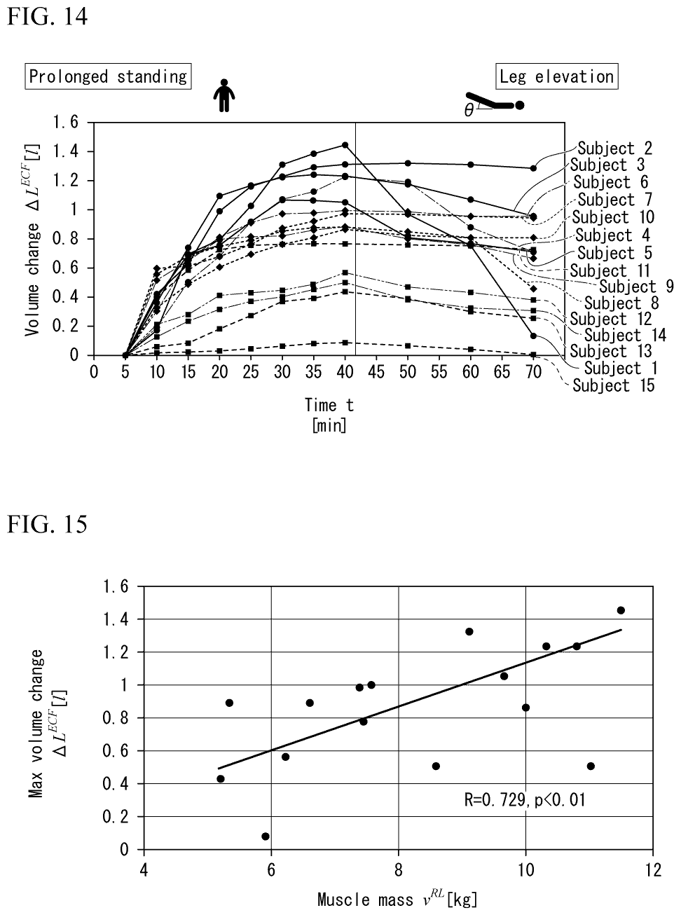

FIG. 14 shows the temporal changes in ΔL^{ECF} of the right legs of 15 subjects. ΔL^{ECF} increased during prolonged standing and ΔL^{ECF} decreased when the legs were raised. Although <Δσ>^{SAT} represents only LSTC, ΔL^{ECF} in FIG. 14 is reasonable based on the assumption that the trend of <Δσ>^{SAT} is consistent with ΔL^{ECF} of the entire right leg.

Next, the range of ΔL^{ECF} was investigated. FIG. 15 shows the correlation between the maximum volume change in ECFmax (ΔL^{ECF}) (ΔL^{ECF} at t⁸=40 minutes), which was measured by bioelectrical impedance analysis of the prior art (conventional BIA) in 15 subjects, and the muscle mass of the right leg v^{RL}. There was a strong positive correlation between max(ΔL^{ECF}) and v^{RL}, for which the score was R=0.729 (p<0.01). Since changes in blood flow are correlated with muscle mass, it is estimated that max (ΔL^{ECF}) due to an increase in intravascular pressure is also correlated with muscle mass.

### Experiment Example 3

In order to quantitatively evaluate the present high-precision EIT method, a comparative examination was conducted between the Example (image reconstruction unit 52+region of interest identification unit 53+learning unit 54: with ROI identification), the Comparative Example (image reconstruction unit 52+learning unit 54: without ROI identification), and the iterative Gauss-Newton method (Equation (43). Gauss-Newton algorithm, Tikhonov) which is the Prior Art Example. Here. Δσ^{iter}ₖ is the conductivity distribution at the iter-th iterating unit at the k-th arbitrary time (1≤k≤3) and η=0.01 is a regularization parameter. The measured impedance ΔZₖ was calculated by FEM calculation similar to the case described above. The element numbers of the forward problem and inverse problem are 3219 and 4261, respectively. The impedance Z was measured at two frequencies, f1=1.6 kHz and f2=4.7 kHz, for which SNR=40 dB due to additive Gaussian noise.

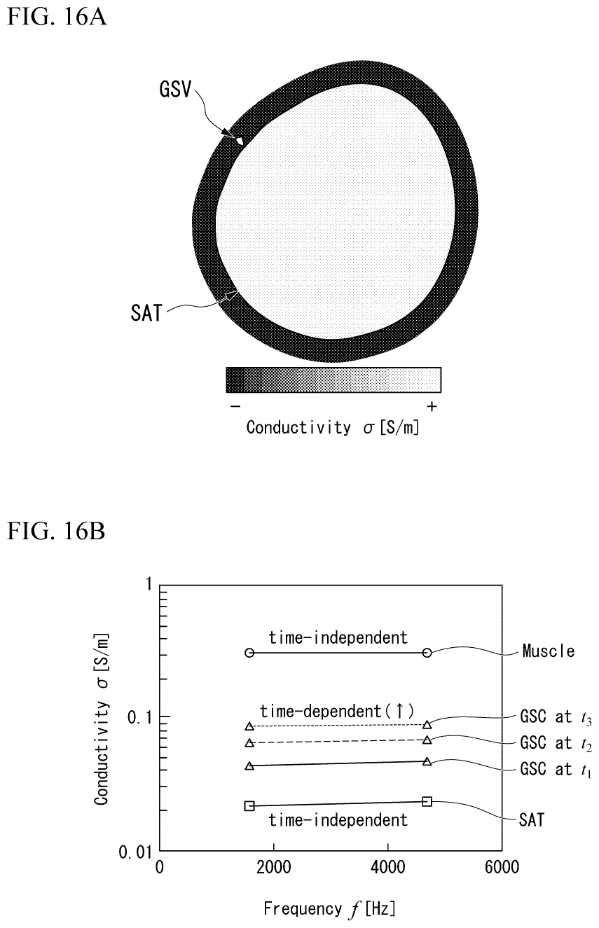

FIG. 16A shows an example of a human calf that includes muscles, SAT, and the GSV having the same boundaries as used in FIG. 11B. FIG. 16B shows the conductivity values a for the simulation. Under a simple unstable background field, only the σ of the GSV is assumed to be time-dependent. At this time, the σ of the SAT and the muscle are assumed to be time-independent and, furthermore, the σ of the muscle is assumed to be frequency-independent. Even in a case where the muscle and SAT are time-independent, image reconstruction of local spatiotemporal changes (LSTC) due to extremely small GSV influence is difficult under frequency-dependent SAT. The σ of the GSV at frequency f1 is 2, 3, and 4 times the σ of any of the t₁-th, t₂-th, and t₃-th SAT, respectively. The σ of the GSV at frequency f2 is adjusted to satisfy Equation (50). In addition, a^{GSV}=0.905 is estimated from experiment data during prolonged standing shown in FIG. 12. This reflected a decrease in the frequency-dependency in ECF a caused by an increase in Na^{+ECF} near the GSV. That is, when σₖ temporarily increases, Δσₖ temporarily decreases. The hyperparameters of the sparse Bayesian learning algorithm were set to regularization parameter β=4 and minimum error range δmin=1×10⁻⁵. The threshold value c of the in-vivo visualization device was set at 75% of the time standard deviation image v and maintained at a higher value. This has a different reference numeral direction from Equation (30). Since c is one of the important parameters that significantly influences the reconstruction result, it is necessary to heuristically optimize c for each application. For example, the LSTC of the target are lost using an inappropriate c. For the maximum number of iterations iter^{max} for all algorithms, itermax=50 was set to examine the changes of various numbers of iterations using the three metrics of the time average localization error <LE> expressed by Equation (51), the spectral error SE expressed by Equation (52), and image noise <IN> expressed by Equation (53). Here, rₖ is the distance between the origin and the center of the reconstructed perturbation Δσₖ^{perturb}b, and <Δσₖ>^{perturb} is the normalized value between the mean 0 and standard deviation 1 of the spatial-mean conductivity <Δσₖ> at the kth arbitrary time inside the perturbation Δσₖ^{perturb}. std(−) is a standard deviation symbol and xₖ is a conductivity distribution that does not belong to Δσₖ^{perturb}. Here, Δσₖ^{perturb} is defined as connected pixels having a conductivity distribution higher than half of the global maximum value. The superscript true indicates the true value of each metric. Under ideal conditions, the values of <LE>, SE, and <IN> are zero.

$$\begin{matrix}[ Equation 12 ] & \\ {Δσ}_{k} = ( {σ}_{k , f 2} - {σ}_{k , f 1} ) + ( k - 1 ) \times ( {σ}_{1 , f 2} - {σ}_{1 , f 1} ) \times {( {a}^{GSV} )}^{k - 1} & ( 50 )\end{matrix}$$

$$〈 LE 〉 = \frac{1}{K} {\sum }_{k = 1}^{K} \vert \frac{{r}_{k} - {r}^{true}}{{r}^{true}} \vert \qquad ( 51 )$$

$$SE = \sqrt{\frac{1}{K} {\sum }_{k = 1}^{K} {( {〈 Δ {\tilde{σ}}_{k} 〉}^{perturb} - {〈 Δ {\tilde{σ}}_{k} 〉}^{true} )}^{2}} \qquad ( 52 )$$

$$〈 IN 〉 = \frac{1}{K} {\sum }_{k = 1}^{K} \frac{std ( {x}_{k} )}{{〈 {Δσ}_{k} 〉}^{perturb}} \qquad ( 53 )$$

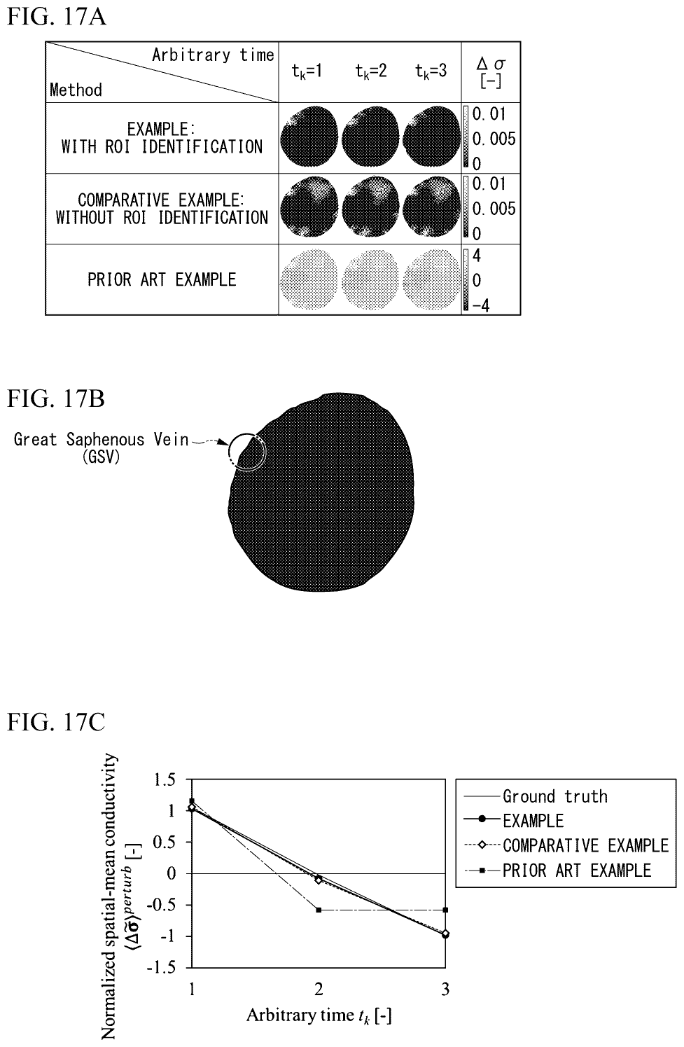

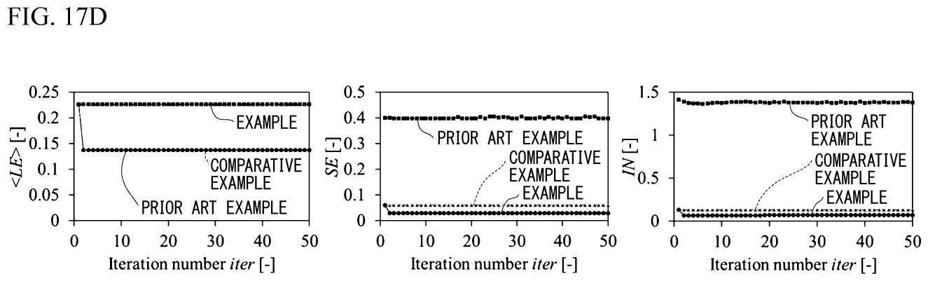

FIG. 17A and FIG. 17C show Δσ at iter=15 which was image-reconstructed by each algorithm under an unstable background field and the corresponding normalized spatial-mean conductivity <Δσ^{˜}k>^{perturb}. FIG. 17B shows an ideal reconstructed image. In addition, the <LE>, SE, and IN of the Example, Comparative Example, and Prior Art Example are shown in FIG. 17D.

Since the reconstruction performance does not change significantly depending on the number of iterations, Δσ at iter=15 is shown. The perturbation Δσₖ^{perturb} which was reconstructed according to the presence of the GSV was distinguished by the sparse Bayesian learning algorithm, while the Gauss-Newton algorithm of the prior art was not able to clearly indicate Δσₖ^{perturh} and had artifacts. Furthermore, <Δσ^{k}>^{perturb} was farthest from a positive value among the algorithms tested. This is considered to be because the Gauss-Newton algorithm simply acquires frequency differences without using temporal correlation or sparsity and therefore is not able to cancel the influence of an unstable background field. On the other hand, in the sparse Bayesian learning algorithm. Δσₖ^{perturh} is clearly distinguished at the correct position. In relation to <Δσ>ₖ^{perturh} in FIG. 17C, although the in-vivo visualization device of the Example was slightly closer to a positive value than the Comparative Example, both estimated the spectrum appropriately. However, artifacts were present in the Comparative Example.

The three algorithms were not significantly dependent on the number of iterations and an SBL algorithm performed better than the Gauss-Newton algorithm. The time average localization error <LE> was the same in the Example and the Comparative Example and the score <LE> was 0.139±0.012. In the case of GSV localization, only iter=2 was sufficient under this condition for both algorithms. The spectral error SE was 0.031±0.004 and 0.061±0.001 in the Example and Comparative Example, respectively. From the above, it was found that the in-vivo visualization device of the Example more appropriately estimates the temporal change in the frequency difference due to the improvement in noise robustness due to SAT separation. The image noise IN was 0.072±0.009 and 0.132±0.001 in the Example and Comparative Example, respectively. In the same manner as the SE results, SAT separation was found to contribute to noise suppression, which is important for monitoring LSTC.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

10: Current/voltage injection measurement unit
52: Image reconstruction unit
53: Region of interest identification unit
54: Learning unit
55: Output unit
100: In-vivo visualization device

What is claimed is:

1. An in-vivo visualization device comprising:
a current/voltage injection measurement unit which has a sensor provided with a plurality of electrodes arrangeable on a subject's skin at intervals from each other and which injects a current or applies a potential difference between each of the electrodes in a state where each of the electrodes is in contact with the skin and measures first measurement data, which is a potential difference and phase, based on a current injection/voltage measurement pattern when injecting the current, or measures second measurement data, which is a current and phase, based on a voltage injection/current measurement pattern when applying the potential difference between each of the electrodes;
an image reconstruction unit which creates an electrical property distribution inside the subject's body based on the first measurement data or the second measurement data and predetermined parameters; and
a region of interest identification unit that performs an identification process on the electrical property distribution to identify a region of interest and creates a post-identification electrical property distribution.

2. The in-vivo visualization device according to claim 1,
wherein the identification process is performed based on a predetermined threshold.

3. The in-vivo visualization device according to claim 1,
wherein the region of interest identification unit creates a region of interest index distribution by performing a standard deviation process on the electrical property distribution and creates the post-identification electrical property distribution by performing the identification process on the region of interest index distribution.

4. The in-vivo visualization device according to claim 2,
wherein the region of interest identification unit creates a region of interest index distribution by performing a standard deviation process on the electrical property distribution and creates the post-identification electrical property distribution by performing the identification process on the region of interest index distribution.

5. The in-vivo visualization device according to claim 1,
wherein the image reconstruction unit creates a multiple measurement matrix from at least one of impedance, resistance, reactance, capacitance, admittance, conductance, susceptance, and phase obtained from the first measurement data or the second measurement data, and
creates the electrical property distribution from the multiple measurement matrix and the predetermined parameters, and
the predetermined parameters include a prior variance vector γ relating to sparsity, a positive definite matrix B relating to temporal correlation, and a variance value λ of a multidimensional normal distribution relating to noise.

6. The in-vivo visualization device according to claim 2,
wherein the image reconstruction unit creates a multiple measurement matrix from at least one of impedance, resistance, reactance, capacitance, admittance, conductance, susceptance, and phase obtained from the first measurement data or the second measurement data, and
creates the electrical property distribution from the multiple measurement matrix and the predetermined parameters, and
the predetermined parameters include a prior variance vector γ relating to sparsity, a positive definite matrix B relating to temporal correlation, and a variance value λ of a multidimensional normal distribution relating to noise.

7. The in-vivo visualization device according to claim 1, further comprising:
a learning unit that updates the predetermined parameters based on at least the post-identification electrical property distribution.

8. The in-vivo visualization device according to claim 7,
wherein the learning unit updates the predetermined parameters by maximizing an average data likelihood using an expectation-maximization algorithm.
